(19) United States
(12) Patent Application Publication
(10) Pub. No.: US 2018/0280204 A1
(43) Pub. Date: Oct. 4, 2018

(54) EXTERNAL FIXATION DEVICE FOR BODY

(71) Applicant: Xieping ZHU, Shanghai (CN)

(72) Inventor: Xieping ZHU, Shanghai (CN)

(21) Appl. No.: 15/790,050

(22) Filed: Oct. 22, 2017

(30) Foreign Application Priority Data
Mar. 28, 2017 (CN) 201720311367.4

(51) Int. Cl. A61F 13/04; A61F 5/02

(52) U.S. Cl. CPC A61F 13/041; A61F 2013/00493; A61F 5/02

(57) ABSTRACT

An external fixation device for body includes a front part and a back part and can be shaped according to the shape of human body. The front part includes a cavity, two layers of knitting cloth, and elastic belts. The back part includes a cavity, two layers of knitting cloth, and plackets. The lower end edge of the front part and the lower end edge of the back part are respectively provided with a filling port and the filling ports are both sewed with a hook and loop. An outer side of one of the elastic belts of the front part and an inner side of one of the plackets of the back part are respectively provided with a piece of a first fastener. An armhole width part of the front part and an armhole width part of the back part are respectively provided with a piece of a second fastener.

### CROSS REFERENCE TO RELATED APPLICATIONS

Pursuant to 35 U.S.C. § 119 and the Paris Convention Treaty, this application claims the benefit of Chinese Patent Application No. 201720311367.4 filed Mar. 28, 2017, the contents of which are incorporated herein by reference. Inquiries from the public to applicants or assignees concerning this document or the related applications should be directed to: Matthias Scholl PC., Attn.: Dr. Matthias Scholl Esq., 245 First Street, 18th Floor, and Cambridge, Mass. 02142.

### BACKGROUND OF THE INVENTION

### Field of the Invention

The invention relates to an external fixation device for fixing body.

### Description of the Related Art

Reduction, fixation and healing are the three steps in treatment of body bone fractures. Typically, the fractures of body bones are treated by adopting tape fixation, traction, internal fixation using stainless steel wire, and external fixation. The materials for external fixation have plaster and prefabricated models. Plaster bandages are not suitable for fixing complicated parts and cannot be taken off if the whole body is fixed. Plaster splints or high polymer splints fail to achieve dynamic close fixation with the constantly changing swelling of patients. Fixation by prefabricated models needs patients to go to hospitals for correction, which wastes time and labor.

### SUMMARY OF THE INVENTION

In view of the above-described problems, it is one objective of the invention to provide an external fixation device for body. The device can be rapidly shaped according to the shape of human body, is suitable for external fixation of a patient in a standing position after surgery and external fixation of a patient in a lying position after surgery, fully meets the patient's treatment requirements, can effectively support and fix fracture parts and shorten treatment and recovery circles.

To achieve the above objectives, in accordance with one embodiment of the invention, there is provided an external fixation device for body that comprises a front part and a back part and can be shaped according to the shape of human body. The front part is in a shape of a Chinese character “ [symbol] ” and is adapted to cover a front part of the body. The back part is in a shape of a Chinese character “ [symbol] ” and is adapted to cover a back part of the body. The front part comprises a cavity, two layers of knitting cloth, and elastic belts. The cavity of the front part is formed between the two layers of knitting cloth. The elastic belts of the front part are respectively sewed on two side seam edges of the front part. The back part comprises a cavity, two layers of knitting cloth, and plackets. The cavity of the back part is formed between the two layers of knitting cloth. The plackets of the back part are respectively sewed on two side seam edges of the back part. The lower end edge of the front part and a lower end edge of the cavity body of the back part are respectively provided with a filling port and the filling port is sewed with a hook and loop. The outer side of one of the elastic belts of the front part and an inner side of one of the plackets of the back part are respectively provided with a piece of a first fastener. The armhole width part of the front part and an armhole width part of the back part are respectively provided with a piece of a second fastener.

When in use, the cavity of the front part and the cavity of the back part are respectively filled with polyurethane slurry through the filling port of the front part and the filling port of the back part; after the polyurethane slurry enters the solid-phase softening stage, the front part and the back part wrap the body of a user through the first fastener and the second fastener, and are shaped according to the shape of the body to fix the body. Doctors and technicians shape the front and back parts to closely fit the body by taking advantage of the special shaping characteristics and memory of the material at the moment. In the coming several minutes, the front and back parts both enter the solid-phase hardening stage to realize rigid fixation.

In some embodiments, two overlapping parts respectively extend from the armhole width part of the front part and the armhole width part of the back part. The two overlapping parts decline to communicate with the cavity of the front part and the cavity of the back part. The second fastener is arranged on the overlapping parts.

In some embodiments, the second fastener is a hook and loop, and comprises two pieces. The two pieces are a hook-type piece and a loop-type piece. The hook-type piece of the second fastener is sewed on an inner surface of the overlapping part of the front part. The loop-type piece of the second fastener is sewed on an outer surface of the overlapping part of the back part.

In some embodiments, the first fastener is a hook and loop, and comprises two pieces. The two pieces are a hook-type piece and a loop-type piece. The hook-type piece is sewed on the outer side of the one of the elastic belts of the front part. The loop-type piece is sewed on the inner side of the one of the plackets of the back part.

The external fixation device for body can be rapidly shaped according to the shape of human body, is suitable for external fixation of patients in a standing position after surgery and external fixation of patients in a lying position after surgery, is convenient in use, can effectively support and fix fracture parts, has the functions of upward and downward dynamic adjustments and frontward and backward dynamic and static adjustments, allows X-rays to penetrate, can fully meet doctors' requirements for treating patients, has a good fixation function, improves and strengthens the fixation quality after surgery and shortens treatment and recovery cycles.

### BRIEF DESCRIPTION OF THE DRAWINGS

FIG. 1 is a right perspective view of an external fixation device for body according to one embodiment of the disclosure; and

FIG. 2 is a left perspective view of an external fixation device for body according to one embodiment of the disclosure.

### DETAILED DESCRIPTION OF THE EMBODIMENTS

The present invention will be further described with reference to the accompanying drawings.

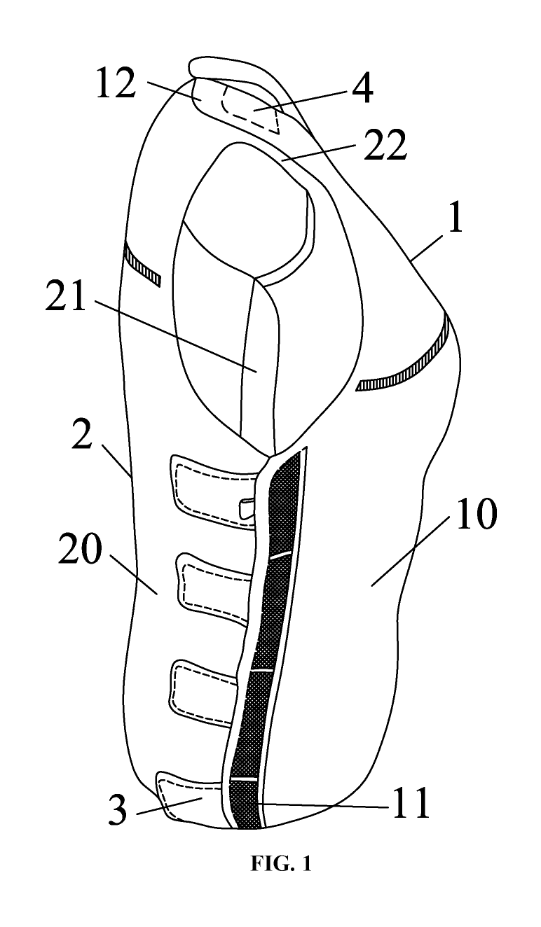

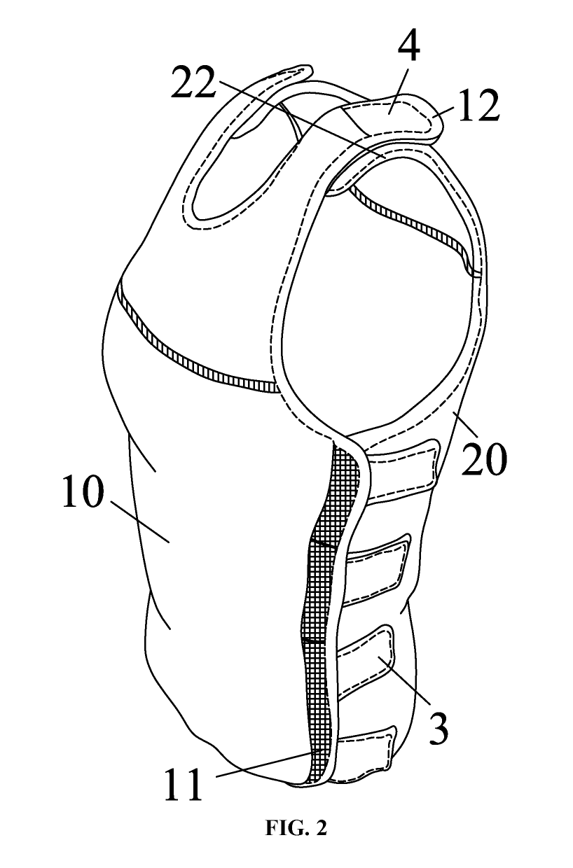

According to FIG. 1 and FIG. 2, an external fixation device for body comprises a front part 1 and a back part 2, and can be shaped according to the shape of human body. The front part is in a shape of a Chinese character “ [symbol] ” and is adapted to cover the front part 1 of the body. The front part 1 comprises a cavity 10 of the front part 1 and elastic belts 11. The cavity 10 of the front part 1 is sewed by two layers of knitting cloth. The elastic belts 11 are respectively sewed on two side seam edges of the front part 1. The back part 2 comprises a cavity 20 of the back part 2 and plackets 21. The cavity 20 of the back part 2 is sewed by two layers of knitting cloth. The plackets 21 are respectively sewed on two side seam edges of the back part 2.The lower end edge of the cavity 10 of the front part 1 and the lower end edge of the cavity 20 of the back part 2 are respectively provided with a filling port and the filling ports are both sewed with a hook and loop.

The outer side of one of the elastic belt 11 of the front part 1 and the inner side of one of the plackets 21 of the back part 2 are respectively provided with a piece of a first fastener 3. The first fastener 3 is a hook and loop, and comprises two, a hook-type piece and a loop-type piece. The hook-type piece is sewed on the outer side of the one of the elastic belts 11 of the front part 1. The loop-type piece is sewed on the inner side of the one of the plackets 21 of the back part 2.

The overlapping parts 12 and 22 respectively extend from the armhole width part of the front part 1 and the armhole width part of the back part 2. The second fastener is arranged on the overlapping parts and the second fastener is a hook and loop, and comprises two pieces, namely a hook-type piece and a loop-type piece. The hook-type piece of the second fastener is sewed on an inner surface of the overlapping part of the front part 1. The loop-type piece of the second fastener is sewed on an outer surface of the overlapping part of the back part 2.

The external fixation device for body is suitable for external fixation of a patient in a standing position after surgery and external fixation of a patient in a lying position after surgery. During use, specially prepared polyurethane A slurry and polyurethane B slurry are mixed to produce polyurethane slurry (the detailed technical scheme is disclosed in the US patent U.S. Pat No. 9,617,370 B2). The cavity 10 of the front part 1 and the cavity 20 of the back part 2 are respectively filled with the polyurethane slurry through the filling port of the front part 1 and the filling port of the back part 2. Then, the polyurethane paste is evened up by being rolled on the operating platform. After the polyurethane slurry enters the solid-phase softening stage, the front part 1 and the back part 2 wrap the body of the patient through the first fastener 3 and the second fastener 4, and are shaped according to the shape of the body. Therefore, the cavity 10 of the front part 1 and the cavity 20 of the back part 2 form a rigid fixation part and can achieve static adjustments and the elastic belt 11 of the front part 1 forms an elastic fixation part which can dynamically and closely fit the body of a patient. The second fastener 4 can be used for larger upward and downward static adjustments, the first fastener 3 can be used for static adjustments for fitting human body sizes and the elastic belt 11 can be used for dynamic adjustments so as to facilitate the automatic close fixation when patients adopt thoracic respiration or diaphragmatic respiration.

In some embodiments, the front part 1 and the back part 2 can be produced into a plurality of specifications. Since the external fixation device for body comprises a rigid fixation part and an elastic fixation part, and is totally different from clinic elastic bandages.

In some embodiments, the external fixation device for body can be rapidly shaped according to the shape of the body, is suitable for external fixation of a patient in a standing position after surgery and external fixation of a patient in a lying position after surgery, is convenient in use, can effectively support and fix fracture parts, has the functions of upward and downward dynamic adjustments and frontward and backward dynamic and static adjustments, allows X-rays to penetrate, can fully meet doctors' requirements for treating the patient, has a good fixation function, improves and strengthens the fixation quality after surgery and shortens treatment and recovery cycles.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. An external device for fixing a body, the device comprising a front part and a back part;
wherein:
the front part is in a shape of a Chinese character “ [symbol] ” and is adapted to cover a front part of the body;
the back part is in a shape of a Chinese character “ [symbol] ” and is adapted to cover a back part of the body;
the front part comprises a cavity, two layers of knitting cloth, and elastic belts;
the cavity of the front part is formed between the two layers of knitting cloth;
the elastic belts of the front part are respectively sewed on two side seam edges of the front part;
the back part comprises a cavity, two layers of knitting cloth, and plackets;
the cavity of the back part is formed between the two layers of knitting cloth;
the plackets of the back part are respectively sewed on two side seam edges of the back part;
a lower end edge of the front part and a lower end edge of the cavity body of the back part are respectively provided with a filling port and the filling port is sewed with a hook and loop;
an outer side of one of the elastic belts of the front part and an inner side of one of the plackets of the back part are respectively provided with a piece of a first fastener;
an armhole width part of the front part and an armhole width part of the back part are respectively provided with a piece of a second fastener; and
the cavity of the front part and the cavity of the back part are respectively filled with polyurethane slurry through the filling port of the front part and the filling port of the back part; in a solid-phase softening stage of the polyurethane slurry, the front part and the back part wrap the body of a user through the first fastener and the second fastener, and are shaped according to the shape of the body to fix the body.

2. The device of claim 1, wherein:
two overlapping parts respectively extend from the armhole width part of the front part and the armhole width part of the back part;
the two overlapping parts decline to communicate with the cavity of the front part and the cavity of the back part; and
the second fastener is arranged on the overlapping parts.

3. The device of claim 1, wherein:
the second fastener is a hook and loop, and comprises two pieces;
the two pieces are a hook-type piece and a loop-type piece;
the hook-type piece of the second fastener is sewed on an inner surface of the overlapping part of the front part; and
the loop-type piece of the second fastener is sewed on an outer surface of the overlapping part of the back part.

4. The device of claim 2, wherein:
the second fastener is a hook and loop, and comprises two pieces;
the two pieces are a hook-type piece and a loop-type piece;
the hook-type piece of the second fastener is sewed on an inner surface of the overlapping part of the front part; and
the loop-type piece of the second fastener is sewed on an outer surface of the overlapping part of the back part.

5. The device of claim 1, wherein:
the first fastener is a hook and loop, and comprises two pieces;
the two pieces are a hook-type piece and a loop-type piece;
the hook-type piece is sewed on the outer side of the one of the elastic belts of the front part; and
the loop-type piece is sewed on the inner side of the one of the plackets of the back part.
